(19) United States
(12) Patent Application Publication
(10) Pub. No.: US 2021/0169250 A1
(43) Pub. Date: Jun. 10, 2021

(54) SMART DISHES AND SMART PACKAGES

(71) Applicants: Roman Czubak, Traiskirchen (AT); Arthur O. Rogers, Las Vegas, NV (US); Tonya Rogers, Las Vegas, NV (US); Peter DeRaedt, Las Vegas, NV (US); Max Lindenberg, Traiskirchen (AT)

(72) Inventors: Roman Czubak, Traiskirchen (AT); Arthur O. Rogers, Las Vegas, NV (US); Tonya Rogers, Las Vegas, NV (US); Peter DeRaedt, Las Vegas, NV (US); Max Lindenberg, Traiskirchen (AT)

(21) Appl. No.: 17/048,969

(22) PCT Filed: Apr. 8, 2019
(86) PCT No.: PCT/US19/26341

§ 371 Date: Oct. 19, 2020

Related U.S. Application Data
(60) Provisional application No. 62/659,512, filed on Apr. 18, 2018.

(51) Int. Cl. A47G 19/02; G01N 33/14

(52) U.S. Cl. CPC A47G 19/02; A47G 2400/123; G01N 33/14

(57) ABSTRACT

A smart dish (10) for food includes a dish (12) having at least one sensor (14) for detecting a predetermined level of contaminate food or beverage contacting the dish (12). A smart package (10) including a package (12) for shipping an object. The smart package (10) includes at least one sensor (14) for detecting a predetermined level of contaminate contacting the package (12).

### CROSS-REFERENCE TO RELATED APPLICATION(S)

The present application claims the benefit of pending Provisional patent application, Ser. No. 62/659,512, filed Apr. 18, 2018.

### BACKGROUND OF THE INVENTION

### 1. Field of the Invention

The present invention relates generally to dishes and packages, more particularly, to smart dishes for food or beverage and smart packages for shipping.

### 2. Description of the Related Art

Every ninety-eight seconds, a person is sexually assaulted. Sometimes this assault is the result of their drinks had been tampered with. Typically, the presence of the common “date rape” drugs GHB and Ketamine is in their drink.

It is known to provide a cocktail stirrer to detect contaminates in a cocktail drink. At the end of the cocktail stirrer, there are two detectors for GHB and Ketamine. Once the stirrer has been placed in the drink, the detectors will detect the drink. If the detector turns to a different color, then the drink has been tampered with and the necessary precautions can be taken.

Currently, these cocktail stirrers are made of glass. This type of construction can result in the stirrer being easily broken. In addition, the stirrer must be cleaned after every use and taken to a place to be used. However, it is desirable to make dishes that are more smart to detect contaminates in food and beverage. It is also desirable to make smart dishes that will alert a person that a contaminate in food or beverage has been detected. It is further desirable to make packages more smart that will detect contaminants contacting the package. Thus, there is a need in the art for new dishes for detecting a predetermined level of contaminate in food or beverage and new packages for detecting a predetermined level of contaminate contacting the package.

### SUMMARY OF THE INVENTION

Accordingly, the present invention provides a smart dish for food or beverage. The smart dish includes a dish having at least one sensor for detecting a predetermined level of contaminate in food or beverage contacting the dish.

In addition, the present invention is a smart package including a package for shipping an object. The smart package includes at least one sensor for detecting a predetermined level of contaminate contacting the package.

One advantage of the present invention is that a new smart dish is provided for food or beverage to detect contaminates in the food or beverage. Another advantage of the present invention is that the smart dish includes a dish having at least one sensor for detecting a predetermined level of contaminate in food or beverage contacting the dish. Yet another advantage of the present invention is that the smart dish can be made of a suitable material such as glass, paper, metal, wood, plastic, or composite material to allow the dish detect a predetermined level of contaminate contacting the dish. Still another advantage of the present invention is that the smart dish can be reused or discarded after use. A further advantage of the present invention is that a new smart package for shipping is provided that includes at least one sensor for detecting a predetermined level of contaminate contacting the package.

Other features and advantages of the present invention will be readily appreciated, as the same becomes better understood, after reading the subsequent description taken in conjunction with the accompanying drawings.

### BRIEF DESCRIPTION OF THE DRAWINGS

FIG. 1 is a plan view of one embodiment of a smart dish, according to the present invention.

FIG. 2 is a sectional view taken along line 2-2 of FIG. 1.

FIG. 3 is a plan view of another embodiment of a smart dish, according to the present invention.

FIG. 4 is a perspective view of yet another embodiment of a smart dish, according to the present invention.

FIG. 5 is a top view of the smart dish of FIG. 4.

FIG. 6 is a perspective view of still another embodiment of a smart dish, according to the present invention.

FIG. 7 is a top view of the smart dish of FIG. 6.

FIG. 8 is a perspective view of a smart package, according to the present invention.

### DESCRIPTION OF THE PREFERRED EMBODIMENT(S)

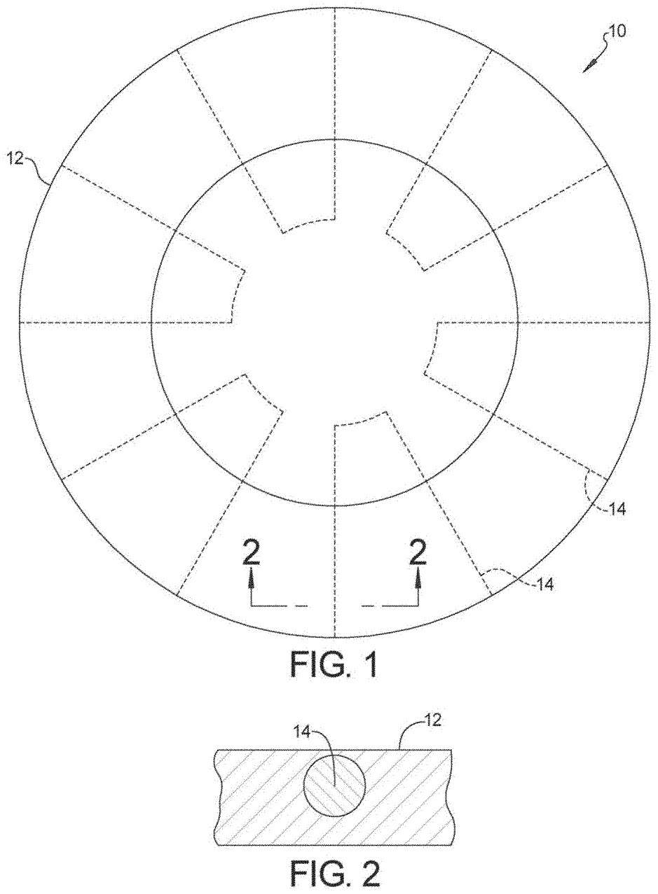

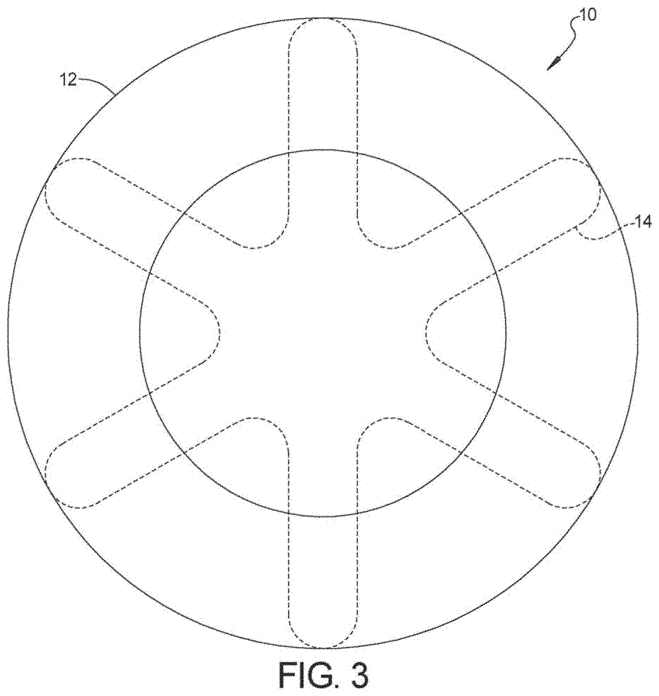

Referring to the drawings and in particular FIGS. 1 and 2, one embodiment of a smart dish, according to the present invention, is generally shown at 10. In the embodiment illustrated, the smart dish 10 includes a dish 12. In one embodiment, the dish 12 is in the form of a plate. The dish 12 is generally circular in shape, but may be any suitable shape. The dish 12 may be made of any suitable material such as glass, paper, metal, wood, plastic, or composite material. The dish 12 is used primarily to hold food. The size and shape of the dish 12 is typically that used in the food and/or consumer industries. It should be appreciated that the dishes 12 are used by human subjects.

As illustrated in FIGS. 1 and 2, the smart dish 10 includes at least one sensor 14. The sensor 14 has a thickness from approximately 2.0 millimeters to approximately 6.0 millimeters, preferably approximately 4.0 millimeters. In one embodiment, the sensor 14 is disposed beneath the surface of the dish 12. In another embodiment, the sensor 14 is disposed on the surface of the dish 12. In one embodiment, the sensor 14 is a plurality of separate and discrete sensors 14 spaced from each other about the dish 12 as illustrated in FIG. 1. In another embodiment, the sensor 14 is one continuous sensor 14 that is disposed in a predetermined pattern about the dish 12 as illustrated in FIG. 3.

The sensor 14 is made of a material whose color changes as a contaminant comes in contact or is sensed. The sensor 14 may, for example, be of an initial color such as white and change successfully from white to another color such as, for example, a deep red when a contaminant has come into contact with the sensor 14. The sensor 14 preferably changes color gradually from white to red passing, for example, through white, red or pink colors before it reaches a deep red color. The extent to which the color red is displayed by the sensor 14 is indicative of the level of contaminants that have cumulatively contacted or sensed by the sensor 14. In another embodiment, the sensor 14 may signal contamination (and its levels) via wireless or other communication mechanism with a user computing device such as a computer, smart phone, tablet, or the like, having a software application that receives and processes the signal. It should be appreciated that a variety of sensors 14 may be used in accordance with the present invention without limitation including, magnetoelastic, microelectromechanical microphysiometer, nanowire, waveguide, liquid crystal, distributed dust or DNA bridge sensors.

A description of each is provided in this paragraph; however, more detailed information of each is readily available in the open literature. A magnetoelastic sensor monitors a change in resonance of a tuned magnetoelastic strip which has been coated with an antibody of the analyte to be detected. The antibodies on the surface of the magnetoelastic strip bond with the analyte when present, changing the mass, and consequently, the resonant frequency of the element which change in mass can be detected to issue a signal. To detect multiple toxins, multiple individual strips may be coated with respective antibodies, ganged together and monitored by a common computer chip for issuing signals. A microelectromechanical sensor monitors changes in the resonance of a spring-mass with a small cantilever beam coated with an antibody of the analyte to be detected to capture a small mass of analyte to effect a change in mass, and, consequently, the resonant frequency of the cantilever beam. A microphysiometer sensor employs live human cells that have been adapted to react quickly to biological agents in the environment. These cells are disposed atop sensors that detect abnormalities in cell structure. Nanowire or DNA bridge sensors employ strings of DNA disposed in or completing an electrical circuit which changes conductivity or resistance as receptors in the DNA molecule accept or combine with other DNA molecules. These DNA strings can be adapted to receive or combine with analyte DNA to detect and issue an alert signal. Waveguide sensors employ a coating of antibodies which are disposed on a sensor surface and selected to target specific analytes such as bacteria cells. When the antibodies come into contact with these bacteria, the antibodies attack and destroy the bacteria and a light source is used to illuminate the changes. As the antibodies destroy the bacteria, the sensor surface detects the changes allowing the bacteria to be identified. Liquid crystal sensors employ cell membranes disposed atop rod-shaped liquid crystals to detect analytes. For example, lipids are attached to the liquid crystals, which lay perpendicular to the surface and appear dark. When the sensor is exposed to a protein that binds to the lipids, the liquid crystal molecules rapidly respond by switching to a planar orientation. As a result, the crystals transmit polarized light and appear bright. The change in illumination can be detected to issue an alert signal. Distributed dust sensors employ micrometer size particles which change color in the presence of contaminate. For example each particle can exhibit different colors depending upon its orientation such that when attaching to a particular contaminate, the particles collectively yield a characteristic optical signature. The change in optical signature can be detected to issue a signal. Immunoassay sensors employ reactive materials which change color or contrast in the presence of an analyte. It should be appreciated that the sensor 14 can includes a white absorptive stick coated with the reactive material which, upon contaminant exposure, effects a color change.

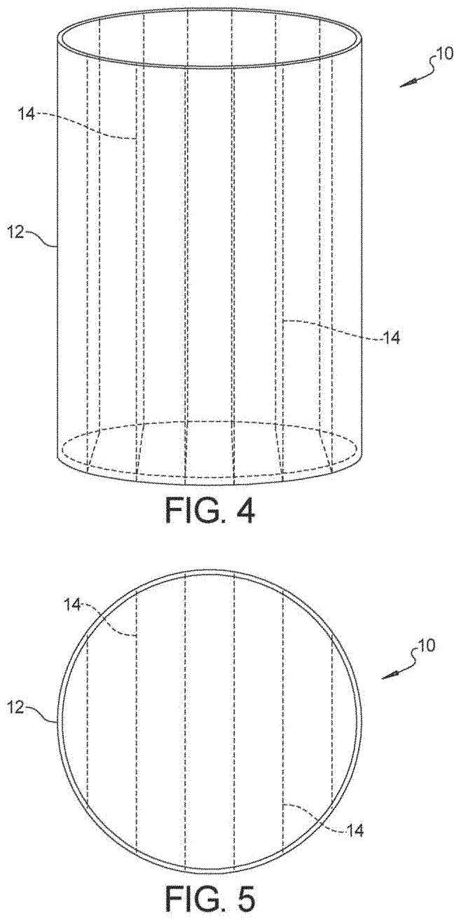

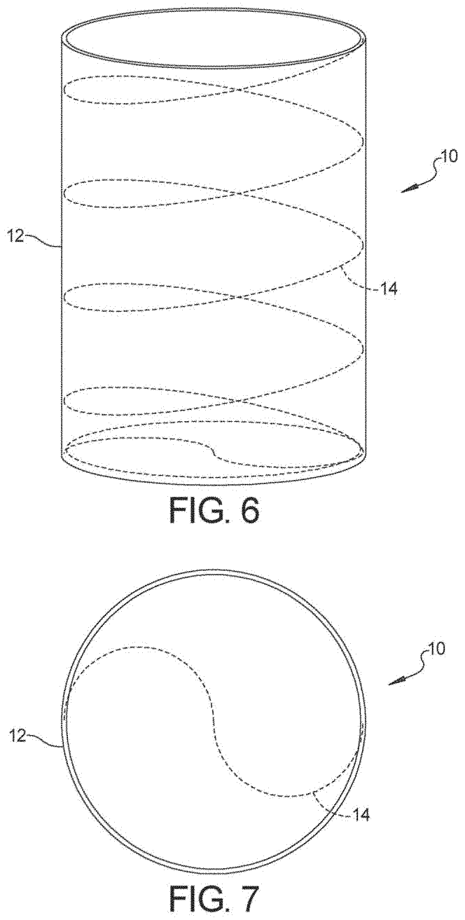

Referring to FIGS. 4-5, another embodiment, according to the present invention, of the smart dish 10 is shown. In this embodiment, the smart dish 10 includes the dish 12 formed as a glass or vessel for holding a liquid beverage. In one embodiment, the sensor 14 is disposed beneath the surface of the dish 12. In another embodiment, the sensor 14 is disposed on the surface of the dish 12. In the embodiment illustrated, the sensor 14 is disposed along the bottom and wall of the dish 12. In one embodiment, the sensor 14 is a plurality of separate and discrete sensors 14 spaced from each other about the dish 12 as illustrated in FIGS. 4 and 5. In another embodiment, the sensor 14 is one continuous sensor 14 that is disposed in a predetermined pattern about the dish 12 as illustrated in FIGS. 6 and 7.

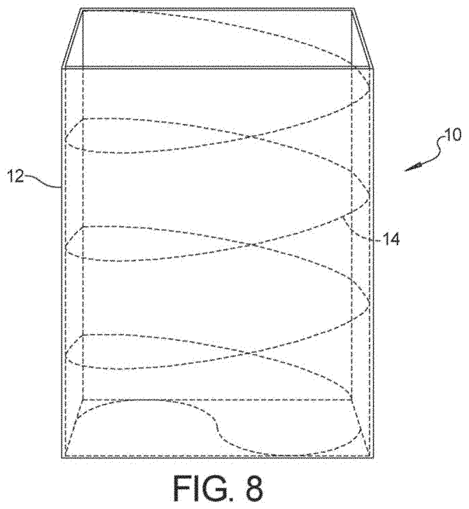

Referring to FIG. 8, one embodiment of a smart package 10, according to the present invention is shown. The smart package 10 includes a package 12 and at least one sensor 14 for detecting a predetermined level of contaminate put on the package 12. In one embodiment, the package 12 may be a box made of any suitable shape, size, and material. In another embodiment, the package 12 may be an envelope made of any suitable shape, size, and material. The sensor 14 has a thickness from approximately 2.0 millimeters to approximately 6.0 millimeters, preferably approximately 4.0 millimeters. In one embodiment, the sensor 14 is disposed beneath the surface of the package 12. In another embodiment, the sensor 14 is disposed on the surface of the package 12. In one embodiment, the sensor 14 is a plurality of separate and discrete sensors 14 spaced from each other about the package 12. In another embodiment, the sensor 14 is one continuous sensor 14 that is disposed in a predetermined pattern about the package 12. The smart package 10 may include a wrap (not shown) made of plastic or foil with one or more sensors 14 adhered to the inside by a suitable mechanism such as an adhesive so that the sensors 14 cannot be removed without damaging the package 12 itself. The smart package 10 may also include a power source (not shown) such as a small flat battery to supply power for a longer period of time to a light emitter (not shown) and to the sensor 14. In one embodiment, the sensor 14 may signal contamination (and its levels) via wireless or other communication mechanism with a user computing device (not shown) such as computer, smart phone, tablet, or the like, having a software application that receives and processes the signal. It should be appreciated that the smart package 10 allows for Bluetooth or Near Field Communication to allow the user computing device to pick up data from the sensor 14.

Accordingly, a method of making the smart dish 10 and the smart package 10 of the present invention includes making the smart dish 10 or the smart package 10 according to the construction of either FIGS. 1-3 or FIGS. 5-7.

The present invention has been described in an illustrative manner. It is to be understood that the terminology, which has been used, is intended to be in the nature of words of description rather than of limitation.

Many modifications and variations of the present invention are possible in light of the above teachings. Therefore, the present invention may be practiced other than as specifically described.

1. A smart dish (10) comprising:
a dish (12) having at least one sensor (14) for detecting a predetermined level of contaminate in food or beverage contacting the dish (12), the at least one sensor (14) being disposed in the dish (12) to signal contamination via wireless or other communication mechanism with a user computing device.

2. A smart dish (10) as set forth in claim 1 wherein said at least one sensor (14) displays a change in color when the contaminate is present.

3. A smart dish (10) as set forth in claim 1 wherein said dish (12) is made of one of glass, paper, metal, wood, plastic, or composite material.

4. A smart dish (10) as set forth in claim 1 wherein said at least one sensor (14) comprises a plurality of separate and discrete sensors spaced from each other about said dish (12).

5. A smart dish (10) as set forth in claim 1 wherein said at least one sensor (14) comprises a one continuous sensor that is disposed in a predetermined pattern about said dish (12).

6. A smart dish (10) as set forth in claim 1 wherein said at least one sensor (14) is disposed on a surface of said dish (12).

7. A smart dish (10) as set forth in claim 1 wherein said at least one sensor is disposed beneath a surface of said dish.

8. A smart dish (10) as set forth in claim 1 wherein said dish comprises a plate to hold food.

9. A smart dish (10) as set forth in claim 1 wherein said dish comprises a vessel to hold a beverage.

10. A smart package (10) comprising:
a package (12) for shipping an object; and
at least one sensor (14) for detecting a predetermined level of contaminate contacting said package (12)), the at least one sensor (14) being disposed in the package (12) with a power source and to signal contamination via wireless or other communication mechanism with a user computing device.

11. A smart package (10) as set forth in claim 10 wherein said at least one sensor (14) displays a change in color when the contaminate is present.

12. A smart package (10) as set forth in claim 10 wherein said package (12) is made of one of glass, paper, metal, wood, plastic, or composite material.

13. A smart package (10) as set forth in claim 10 wherein said at least one sensor (14) comprises a plurality of separate and discrete sensors spaced from each other about said package (12).

14. A smart package (10) as set forth in claim 10 wherein said at least one sensor (14) comprises a one continuous sensor that is disposed in a predetermined pattern about said package (12).

15. A smart package (10) as set forth in claim 10 wherein said at least one sensor (14) is disposed on a surface of said package (12).

16. A smart package (10) as set forth in claim 10 wherein said at least one sensor (14) is disposed beneath a surface of said package (12).

17. A smart package (10) as set forth in claim 10 wherein said package (12) comprises a box to contain the object.

18. A smart package (10) as set forth in claim 10 wherein said package (12) comprises an envelope to contain the object.
